(19) United States
(12) Patent Application Publication
(10) Pub. No.: US 2015/0064270 A1
(43) Pub. Date: Mar. 5, 2015

(54) COMPOSITION AND METHOD FOR EFFECTIVE LEAN BODY MASS GAIN

(71) Applicant: PROLIANT INC., Ankeny, IA (US)

(72) Inventor: Eric M. Weaver, Story City, IA (US)

(21) Appl. No.: 14/454,385

(22) Filed: Aug. 7, 2014

Related U.S. Application Data
(63) Continuation of application No. 11/584,251, filed on Oct. 20, 2006, now abandoned.
(60) Provisional application No. 60/728,938, filed on Oct. 22, 2005.

(51) Int. Cl. A61K 35/20; A61K 9/00; A61K 35/16

(52) U.S. Cl. CPC A61K 35/20; A61K 35/16; A61K 9/0095
USPC 424/531

(57) ABSTRACT

This invention provides compositions comprising serum protein isolate, whey protein concentrate, and whey protein isolate. The invention also provides methods for increasing lean body mass in a human, comprising administering to the human an effective amount of a composition comprising serum protein isolate and whey protein concentrate.

### CROSS-REFERENCE TO RELATED APPLICATION(S)

This application claims the benefit of U.S. Provisional Application No. 60/728,938, filed on Oct. 22, 2005, under 35 U.S.C. §119(e), which is hereby incorporated by reference.

### BACKGROUND

The dietary protein requirement of human beings has been thoroughly researched and estimates of the requirement established (First joint FAO/WHO Expert Consultation on Energy Intake and Protein Requirements, Food Nutr (Roma.) 3:25 (1977)). Additionally, many factors are known to affect the amount of protein necessary to meet the body's protein requirement including age, gender, lean body mass, exercise, and dietary factors. Protein quality is one factor that affects the quantity of dietary protein necessary to meet the body's protein requirement (Young, V. R., et al. J Nutr 105:534-42 (1975); Garza, C., et al., Am J Clin Nutr 29:280-7 (1976); Walberg, J. L., et al., Int. J Sports Med 9:261-6 (1988)). The determination of protein quality incorporates measures of amino acid composition and digestibility as well as the ability of a particular protein source to promote growth.

Physical activity and training are also important factors affecting the dietary protein requirement. Both endurance exercise and resistance training are associated with dietary protein needs greater than the current Recommended Dietary Allowance (RDA) for adults (Lemon, P. W., Nutr Rev. 54:S169-S175 (1996); Evans, W. J., Can. J Appl Physiol 26 Suppl:S141-S152 (2001); Tarnopolsky, M., Nutrition 20:662-8 (2004)). The protein requirements for maximal lean body mass from a mixed protein diet are estimated to be 1.2-1.4 g per kg of body weight (BW) for endurance training and 1.7-1.8 g protein per kg BW for strength training (Evans, W. J., Can. J Appl Physiol 26 Suppl:S141-S152 (2001); Tamopolsky, M., Nutrition 20:662-8 (2004); Lemon, P. W., Nutr Rev. 54:S169-S175 (1996)). The increased protein requirements of athletes in training (Tarnopolsky, M. A., J Appl Physiol 64:187-93 (1988)) has provided them with the motivation to select high quality proteins for supplementation.

Thus, athletes in training often consume protein supplements as part of their diet. The dietary consumption of high quality proteins is known to both increase lean body mass and to reduce muscle soreness in trained athletes (Flakoll, P. J., et al. J Appl. Physiol 96:951-6 (2004)). While the specific essential amino acid requirements for increased physical activity are not well-established, the main function of high quality proteins is to provide essential amino acids for maximizing lean body mass in an athlete. Milk, whey, colostrum, egg, and soy protein are all commonly available, high quality proteins that can be used in protein supplements. The carbohydrates and minerals are removed from these protein sources in order to concentrate these proteins. The resulting protein products are typically spray-dried as protein concentrates, which typically contain more than 80% protein on a dry-basis, or as protein isolates, which typically contain more than >90% protein on a dry basis. Further processing of these protein sources, such as by denaturation or enzymatic hydrolysis, are sometimes used to improve digestibility or absorption of amino acids. Essential amino acids can also be supplied using synthetic amino acids produced by fermentation or chemical synthesis. Essential amino acids such as L-lysine, L-isoleucine, L-leucine, and L-valine and L-arginine are often included in protein supplements to increase the dietary levels of these specific essential amino acids. Higher levels of each of these amino acids are believed to be required to maintain or increase muscle mass during physical training.

In addition to meeting the essential amino acid requirements, a few non-essential amino acids, such as creatine and glutamine, have been found to be beneficial to athletes in training. Other substances, such as calcium, pyruvate, and conjugated linoleic acid, are used to enhance training or performance. As a result, blends of proteins and amino acids are often used to provide for an amino acid profile that may more closely meet the estimated amino acid requirements of the athlete. Even with increased levels of high quality protein and/or amino acid intake, experienced, trained athletes commonly reach a plateau in which they experience minimal gains in lean body mass in response to supplementation.

Anabolic peptides, such as IGF-I, insulin, TGF-β and growth hormone, and other compounds such as like phospholipids and conjugated linoleic acid, are all found in milk or serum. These compounds are known to promote protein synthesis and increases in lean body mass. However, the low concentration of these compounds in milk does not provide much benefit in building lean body mass. Also, the concentration of these compounds in whey protein concentrates, dried skim milk or other milk protein products is normally even lower because of damage or removal by the usual processing procedures of pasteurization and/or isolation of protein.

Further, the capacity of bioactive peptides such as IGF-I and TGF-β to promote protein synthesis after oral administration has not been demonstrated when each growth factor is given individually (Donovan, S. M., J Pediatr. Gastroenterol. Nutr 24:174-82 (1997)). There may be potential for protein synthetic activity and the development of muscle if they are consumed at sufficient levels in preparations containing multiple peptides, such as they are when they are included in serum in vivo (Doumit, M. E., J Cell Physiol 157:326-32 (1993)).

Whey protein, also known in the art as lactoserum or lactalbumen, is recognized in the art as a source of protein that can increase growth rate and treat disease in animals when administered orally or through dietary supplementation (Stott, G. and Lucas, 0., U.S. Pat. No. 4,834,974). Bounous et al. teaches that the oral supplementation of undenatured whey protein concentrate can be used to enhance active immunity in mice (Bounous, G., et al., U.S. Pat. No. 5,451,412). Contrary to the needs of an athlete, increasing immune system activity will likely increase inflammatory cytokine production, which in turn will reduce lean tissue accretion. Gardiner et al. teaches that combining a nitric oxide precursor with whey protein will increase lean body mass more than only whey protein or the placebo (Gardiner, P. T., et al., U.S. Pat. No. 6,784,209). U.S. Pat. No. 6,221,836 discloses an anabolic protein composition designed to mimic the amino acid profile of human muscle tissue. WO Patent 99/56758 discloses the use of a food supplement containing undenatured colostrum, colostral-derived growth factors and colostrum-derived casein to change body composition (Whyte, P. B. WO9956758).

Lastly, stress hormones and cytokines are factors known to modulate gains in lean body mass in animals. Campbell et al. (U.S. patent application Ser. No. 10/470,982) identified a plasma protein composition that modulates the immune system of animals.

What is needed are improved protein-based compositions and methods that promote gains in lean body mass and rapid recovery from intensive training in the trained athlete.

### SUMMARY OF THE INVENTION

The present invention provides compositions comprising serum protein isolate and whey protein concentrate. The present invention also provides methods for increasing lean body mass in a human, comprising administering to the human a composition comprising effective amounts of serum protein isolate and whey protein concentrate. The compositions of the present invention optionally comprise whey protein isolate.

The present invention further provides methods for increasing lean body mass in a human, comprising administering to the human a composition comprising effective amounts of serum protein isolate, microfiltered whey protein concentrate, and whey protein isolate.

The present invention provides methods for inhibiting inflammation in a human, comprising administering to the human a composition comprising effective amounts of serum protein isolate, microfiltered whey protein concentrate, and whey protein isolate.

The present invention also provides methods for reducing muscular soreness in a human, comprising administering to the human a composition comprising effective amounts of serum protein isolate, microfiltered whey protein concentrate, and whey protein isolate.

In one embodiment of the present invention, the composition comprises serum protein isolate and whey protein concentrate. In another embodiment of the present invention, the composition further comprises whey protein isolate.

In one embodiment of the present invention, serum protein isolate is present in at least about 1% of the composition. In another embodiment of the present invention, serum protein isolate is present in at least about 5% of the composition.

In one embodiment of the present invention, whey protein concentrate is present in at least about 50% of the composition. In a further embodiment of the present invention, whey protein concentrate is present in at least about 70% of the composition.

In one embodiment of the present invention, the whey protein concentrate is microfiltered.

In one embodiment of the present invention, the whey protein isolate is present in at least about 5% of the composition. In another embodiment of the present invention, the whey protein isolate is present in at least about 10% of the composition.

In another embodiment of the present invention, the compositions further comprise a solubility agent.

In one embodiment of the present invention, the compositions further comprise a flavoring agent.

In one embodiment of the present invention, serum protein isolate is derived from bovine, porcine or ovine serum. In another embodiment of the present invention, whey protein isolate is prepared from the milk of a cow, a goat, or a sheep.

In one embodiment of the present invention, method is provided for increasing lean body mass in a human, comprising administering to the human an effective amount of a composition comprising serum protein isolate and whey protein isolate.

In another embodiment of the present invention, method is provided for increasing lean body mass in a human, comprising administering to the human an effective amount of a composition comprising serum protein isolate, microfiltered whey protein concentrate, and whey protein isolate.

In yet another embodiment of the present invention, method is provided for inhibiting inflammation in a human, comprising administering to the human an effective amount of a composition comprising serum protein isolate, microfiltered whey protein concentrate, and whey protein isolate.

In one embodiment of the present invention, method is provided for reducing muscular soreness in a human, comprising administering to the human an effective amount of a composition comprising serum protein isolate, microfiltered whey protein concentrate, and whey protein isolate.

In one embodiment of the present invention, the human is a conditioned athlete.

In another embodiment of the present invention, the increase in lean body mass provided by the composition is greater than the increase in lean body mass provided by an equal amount of the whey protein concentrate administered similarly.

### DETAILED DESCRIPTION OF THE INVENTION

The invention is described with reference to various specific embodiments and techniques, however, it should be understood that these specific embodiments and techniques are non-limiting, and that many variations and modifications may be made while remaining within the scope of the invention.

Existing protein supplement products designed for increasing lean body mass and inhibiting inflammation in humans do not contain the combination of milk and serum-derived fractions, as is provided by the present invention. The compositions of the present invention optimize lean body mass and modulate inflammatory cytokine production in humans.

A few products are commercially available which contain higher levels of growth factors, conjugated linoleic acid, and phosphatidylserine. One example of such a product is the lipid-rich retentate of whey protein isolate processing. However, this product has not been used in protein supplement formulations designed to increase lean body mass. In addition, plasma and serum protein concentrates and isolates, though they contain high levels of some of these proteins and growth factors, are not used in protein supplement formulations designed to increase lean body mass.

The compositions of the present invention increase fat-free lean body mass in trained athletes to a greater degree than the consumption of protein concentrate products or protein isolate products based on whey, casein, egg, soy and combinations thereof, where the products are greater than 80% protein on a dry matter basis.

This compositions of the present invention comprise isolated serum proteins, a microfiltered whey concentrate, and a minimum effective amount of whey protein isolate which provides a composition of bioactive proteins and other factors that is unexpectedly more effective in inhibiting inflammation and increasing lean body mass than whey protein concentrate or isolates used alone or administered singly.

The inventors unexpectedly discovered that greater improvements in lean body mass can be obtained by increasing the levels of growth factors, immunoglobulin, conjugated linoleic acid, and phosphatidylserine content in a protein supplement.

The phrases “trained athlete,” “conditioned athlete,” and the like are used interchangeably herein. The phrases “trained athlete” and “conditioned athlete” as used herein describe a physically fit human being who has exercised and/or trained his or her body to develop the strength, agility, and endurance that are necessary for intense physical exercise or sport.

The phrase “whey protein isolate” (or WPI) as used herein, refers to whey protein containing at least 90% protein on a dry matter basis. WPI comprises protein, fat, lactose, immunoglobulins, growth factors (IGF-1, TGF-B1, TGF-B2), sphingomyelin, phospholipids, minerals, and moisture. See Table I for a list of selected active components of WPI. The compositions of the present invention comprise at least about >5 to 15% WPI. WPI is available for purchase from commercial producers of whey protein. For example, IsoChill™ 6000 from Trega Foods (Weyauwega, Wis., U.S.A.).

The phrases “microfiltered whey protein concentrate,” or “whey protein retentate,” “microfiltered whey protein concentrate,” or “whey protein concentrate,” (WPC) as used herein, refer to a high-lipid liquid that is a co-product of whey protein isolate processing. WPC is a liquid concentrate comprising lipids and liquids that are retained on the microfiltration module during the filtration process in the manufacturing process of whey protein isolate. The microfiltration process is either a cross-flow or perpendicular microfiltration. After the WPC is collected, it is spray-dried. WPC contains about 10-30%, or about 15-25%, or about 18-20% solids prior to drying. The dried WPC contains a concentrated source of phosphatidyl serine, phosphatidyl choline, phosphatidyl ethanolamine, conjugated linoleic acid, lactoferrin, growth factors, and whey proteins such as β-lactoglobulin and α-lactalbumen. (See Table I for a list of selected active components of WPC.) The compositions of the present invention comprise at least about 60 to 80% WPC. WPC is available for purchase from commercial producers of whey protein isolates, and is generally sold under trade name as a whey protein product. For example, Salibra®702 from Glanbia (Monroe, Wis., U.S.A.).

As used herein, the phrase “serum protein isolate” (or SPI), refers to a concentrated source of albumin, immunoglobulins, growth factors, and amino acids. SPI contains protein, water, ash, fat, carbohydrates, lactose, albumin, immunoglobulin G, calcium, chloride, magnesium, phosphorus, potassium, sodium, alanine, arginine, aspartic acid, cystine, glutamic acid, glycine, histidine, isoleucine, leucine, lysine, methionine, to phenylalanine, praline, serine, threonine, tryptophan, tyrosine, and valine. The compositions of the present invention comprise at least about 2 to 10% SPI. As used herein, SPI is generally derived from bovine, porcine or ovine serum. SPI is available from Proliant Health Ingredients, Ankeny, Iowa, U.S.A. (ImmunoLin or NutraGammax)

Solubility and flavoring agents may be added to the compositions of the present invention. Such solubility and flavoring agents include, but are not limited to rice syrup solids, sucrose, phosphatidylcholine, natural and artificial flavors, aspartame, sucralose, and other processing aids. Additionally, syrup solids of grains, including rice and corn, may be added to the compositions of the present invention.

Amino acids and many other substances may also be added to the protein formulation to enhance the effectiveness of the protein composition for specific training or sports activities. For example, creatine may be beneficial for resistance training. Amino acids that may be added include, but are not limited to, L-alanine, L-arginine, L-aspartamine, L-cystine, L-glutamine, L-isoleucine, L-leucine, L-lysine, L-methionine, L-phenylalanine, L-proline, L-serine, L-threonine, L-tryptophan, L-tyrosine, and L-valine. Additionally, vitamins, minerals and other substances may be used to improve other nutritional aspects, such as antioxidants, calcium sources, or B vitamins.

Flavoring agents may be added to the compositions of the present invention. Flavoring agents useful in the present invention may include a fruit flavor, vegetable flavor, chocolate flavor, vanilla flavor, soft drink flavor, or malt flavor. The compositions of the present invention can be formulated for administration to humans, in a variety of forms adapted for oral consumption.

Preferably, the compositions of the present invention can be formulated as a dry mix, which may be blended or shaken with liquids such as milk, water, or juice to make a beverage as a solution or suspension. The compositions of the present invention may also be formulated as an edible part of a food vehicle, such as a bar product. The compositions of the present invention may also be formulated as part of an edible confectionary product, such as a chewable candy-type product. The present compositions may be orally administered in combination with an acceptable vehicle such as an inert diluent or an assimilable edible carrier.

They may be enclosed in hard or soft shell gelatin capsules, may be compressed into tablets, or may be incorporated directly with the food of the diet of the human. For oral administration, the compositions may be combined with one or more excipients and used in the form of ingestible tablets, buccal tablets, troches, capsules, elixirs, suspensions, syrups, wafers, extruded food bars, and the like.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be acceptable for oral consumption and substantially non-toxic in the amounts employed. In addition, the compositions may be incorporated into sustained-release preparations and devices.

In some embodiments of the present invention, the methods of preparation are vacuum drying and freeze drying techniques, which yield a powder of the composition plus any additional desired ingredient present in the previously sterile-filtered solutions.

The blending of the components of the present composition may be accomplished by methods commonly used in the industry, including, but not limited to, paddle, ribbon, and v-blenders. The compositions of the present invention may be blended, agglomerated, or blended and agglomerated to improve the dispersability of the product in solution.

The compositions of the present invention are prepared by dry blending the appropriate concentrations of WPC and SI. The compositions of the present invention preferably comprise 50-80% WPC and 1-10% SPI. The remaining percentage of the compositions of the present invention optionally may comprise 5-15% WPI, and 7-15% non-active ingredients, such as flavoring agents, solubility agents, amino acids, vitamins, minerals, stabilizing agents and the like. Table 1 contains a listing of selected active components of the compositions of the present invention, and of WPC and WPI.

TABLE 1
Selected Active Components of WPC, WPI and Compositions
of the Invention (Expressed as per 100 g protein)

| Selected | Whey protein | | Whey protein | | | |
|---|---|---|---|---|---|---|
| active | concentrate | | isolate | | | |
| components | (WPC) | | (WPI) | | Invention | |
| Growth factors | 17,000 | ng/g | 24,000 | ng/g | >75,000 | ng/g |
| (IGF-I, TGF-B) | | | | | | |
| Immunoglobulin | 3-5 | g | 5-8 | g | >10 | g |
| Lactoferrin | 163 | mg | 132 | mg | >500 | mg |

| | | | | | |
|---|---|---|---|---|---|
| Phosphatidylserine | 60 | mg | Trace | >100 | mg |
| Phosphatidylcholine | 240 | mg | Trace | >350 | mg |
| Conjugated | 11 | mg | Trace | >15 | mg |
| linoleic acid | | | | | |

The compositions of the present invention comprise 2-10 gm of fat, 20-45 mg cholesterol, 70-100 mg sodium, 125-250 mg potassium, 0-10 gm dietary fiber, 0.5-10 gm sugars, 1-10 gm total carbohydrate, 25-60 gm protein. The compositions of the present invention further comprise vitamin A (1-10% of the daily value), vitamin C (0-7% of the daily value), calcium (10-50% of the daily value), iron (1-10% of the daily value), and phosphorus (12-40% of the daily value). (Note that the percentage of daily values are based upon a 2,000 calorie per day diet).

The formulations of the compositions of the present invention comprise at least about 50-80% WPC, 5-15% WPI, 1-10% SPI, and 8-15% of the combination of non-active ingredients, including sweeteners (such as syrup solids, sucrose, aspartame, acesulfame potassium), flavoring agents, and dispersion agents, such as soy lecithin. Preferably, the net amount of protein in the compositions of the present invention is at least about 65-80%. In another embodiment of the present invention, the formulations of the compositions comprise at least about 70-75% WPC, 7-11% WPI, 3-6% SPI, and 10-13% non-active ingredients. In preferred embodiment of the invention, each dose contains the active components at a ratio of about 10-15 (WPC): 1.5-4.0 (WPI): 0.5-1.5 (SPI) wt/wt.

A preferred dosage form should contain at least between a 10 to 60 gram dose of the composition, formulated as a dry mix. Another preferred dosage form should contain at least between a 25 to 55 gram dose of the composition, formulated as a dry mix. Another preferred dosage form should contain at least about 50 grams of the composition, formulated as a dry mix. The percentage of the compositions may, of course, be varied and may conveniently be between about 25 to about 95% of the weight of a given unit dosage form. The amount of active composition in such formulations and preparations is such that an effective dosage level will be obtained.

In one embodiment of the present invention, an effective dose of the active composition will be from 0.250 to 2.0 grams of the composition per kilogram body weight per day. In one embodiment of the present invention, the effective amount of the composition is from about 0.1 to 10 grams per kilogram of body weight of per day.

In another embodiment of the present invention, an effective dose of the active composition will be from about 0.50 to 1.0 grams of the composition per kilogram body weight per day. In another embodiment of the present invention, an effective dose of the active composition will be from about 0.5 to 5 grams of the composition per kilogram body weight per day. In another embodiment of the present invention, an effective dose of the active composition will be from about 0.75 to 2.0 grams of the composition per kilogram body weight per day.

In another embodiment of the invention, the effective does of the active compound will be about 0.15 to 0.35 gram per pound of body weight. In another embodiment of the invention, the effective dose of the active compound will be about 0.5 to 0.80 gram per pound of body weight. In yet another embodiment of the invention, the effective dose of the active compound will be about 0.75 to 1.0 gram per pound of body weight.

In another embodiment, the invention provides an ingestible composition comprising 70-80% whey protein concentrate, 5-15% whey protein isolate, 1-7% serum protein isolate, and 10-15% non-active ingredients. In another embodiment, the ingestible composition is formulated as a dry mixture.

In an embodiment of the present invention, the composition is administered in two or more doses per day. In another embodiment of the present invention, the composition is administered in one dose per day.

### EXAMPLES

In the Examples below, the participants consumed 50 gm doses of the composition of the present invention. Each 50 gram dose was in a dry mix form, and was reconstituted in 8-12 oz. of water and consumed orally. Each 50 gm dose contained 37 gm WPC, 5 gm WPI, 2 gm SPI, and 6 gm non-active ingredients (sweeteners, flavors and soy lecithin).

### Example I

A 26-year old human male, who weighed 160 pounds, was given three 50 gram doses of the composition of the invention daily over a period of 3.5 weeks. The participant was a professional trainer and sports condition specialist with a 10 year history of whey protein supplementation and training. During the period of supplementation, the participant was in an intensive weight lifting training program. At the beginning of the supplementation period, the participant was able to bench press one set of five repetitions at 275 lbs.

At the end of the 3.5 weeks, the participant's lean body mass increased nine pounds over the participant's amount of lean body mass prior to beginning consumption of the composition of the invention. The participant was able to complete three sets of five repetitions pressing 275 lbs. Additionally, the participant reported a reduction in muscle soreness to one day of muscle soreness instead of the usual 3 days.

### Example II

A 27-year old human male was given three 50 gram doses of the composition of the invention daily, over a period of 10 weeks. The participant was an amateur athlete.

At the end of the 10 weeks, the participant gained 7 pounds of lean body mass over the participant's amount of lean body mass prior to beginning consumption of the composition of the invention. The participant reported gains in strength, an increased ability to train longer, and no muscle soreness despite an increase in resistance training.

### Example III

A 36-year old human male, who measured 6 feet, 1 inch and weighed 206 pounds, was given three 50 gram doses of the composition of the invention daily, for 180 days. The maximum amount of weight that the participant was able to bench press was measured prior to beginning the experiment.

At the end of the 180 days, the participant gained 12 pounds of lean mass over the participant's amount of lean body mass prior to beginning consumption of the composition of the invention. The participant's bench press maximum increased by 40 pounds. Additionally, the participant reported a significant reduction in post-workout soreness, which allowed for longer workouts and an increase in strength.

### Example IV

A 47-year-old female was given three 50 gram doses of the composition of the invention per day for 40 days. The maximum amount of time that the participate was able to aerobically exercise was measured prior to beginning the experiment.

At the end of the 40 days, the participant reported an increased level of energy, an increased ability to train, and a reduction in appetite. The participant was also able to increase the duration of aerobic exercise by 45 minutes.

### Example V

A 41-year-old, 5 feet, 9 inch male who weighed 162 pounds, was given one 50 gram dose of the composition of the invention each day for 70 days. The man had a five year history of aerobic and light resistance training.

At the end of the 70 days, the participant gained 4 pounds of lean body mass over the participant's amount of lean body mass prior to beginning consumption of the composition of the invention. There was no change in the participant's body fat measurement. Additionally, the man reported significantly reduced post-workout muscle soreness when a different resistance exercise was introduced.

After the participant achieved the above-recited results, he was given supplementation with a 100% whey protein product (ProPerformance 100% Whey Protein Product, General Nutrition Centers, Inc. U.S.A.), at 31 gram/dose, two doses per day, for 42 days. At the end of the 42 days, the participant had lost 4 pounds of lean body mass, and the muscle soreness returned to that experienced prior to the beginning of Example V.

All publications, patents and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1-2. (canceled)

3. The method of claim 14, wherein the serum protein isolate is present in about 5% of the composition.

4. The method of claim 14, wherein the whey protein concentrate is present in at least about 70% of the composition.

5. (canceled)

6. The method of claim 14, wherein the whey protein isolate is present in about 10% of the composition.

7. The method of claim 14, wherein the composition further comprises a solubility agent.

8. The method of claim 14, wherein the composition further comprises a flavoring agent.

9. The method of claim 14, wherein the serum protein isolate is derived from bovine, porcine or ovine serum.

10. The method of claim 14, wherein the whey protein isolate is prepared from the milk of a cow, a goat, or a sheep.

11-13. (canceled)

14. A method for reducing post-exercise muscle soreness in a human, comprising administering to the human an effective amount of a composition comprising about 2 to 10% serum protein isolate, at least 50% microfiltered whey protein concentrate, and about 5 to 15% whey protein isolate, so that post-exercised soreness is reduced, wherein the effective amount is from about 0.1 to 10 grams per kilogram of body weight per day.

15. (canceled)

16. The method of claim 14, wherein the effective amount of the composition is from about 0.1 to 10 grams per kilogram of body weight of per day.

17. The method of claim 14, wherein the effective amount of the composition is from about 0.75 to 2.0 grams per kilogram of body weight of per day.

18-19. (canceled)
